(19) United States
(12) Patent Application Publication
(10) Pub. No.: US 2008/0214435 A1
(43) Pub. Date: Sep. 4, 2008

(54) Novel Cancer Indications of Mannan-Binding Lectin (Mbl) in the Treatment of Immunocompromised Individuals

(75) Inventor: Martin Bonde, Lyngby (DK)

Correspondence Address: BROWDY AND NEIMARK, P.L.L.C.;624 NINTH STREET, NW, SUITE 300, WASHINGTON, DC, 20001-5303, US

(73) Assignee: Natlmmune A/S, Copenhagen (DK)

(21) Appl. No.: 11/911,262

(22) PCT Filed: Apr. 6, 2006
(86) PCT No.: PCT/DK2006/000197

§ 371 Date: Oct. 11, 2007

(30) Foreign Application Priority Data
Apr. 11, 2005 (DK) PA 2005 00508

(51) Int. Cl. A61K 38/02; A61P 31/04; A61P 31/12

(52) U.S. Cl. 514/2

(57) ABSTRACT

The present invention pertains to the use of subunits and oligomers of mannan-binding lectin (MBL) in prophylactic and/or curative treatment of an immunocompromised individual such as subjects suffering from solid tumors or haematological cancers. Solid tumors include such as female cancers, male cancers, cancers of the respiratory system, cancers of the gastro intestinal system, the renal system and further subjects suffering from thyroid cancer and melanomas. Haematological cancers include leukaemia, lymphoma and myeloma. The immunocompromised condition of the individual may be due to a cancer disease as mentioned herein or the treatment of said cancer disease.

### TECHNICAL FIELD

The present invention pertains to the use of subunits and oligomers of mannan-binding lectin (MBL) in prophylactic and/or curative treatment of an immunocompromised individual such as subjects suffering from solid tumors or haematological cancers. Solid tumors include such as female cancers, male cancers, cancers of the respiratory system, cancers of the gastro intestinal system, the renal system and further subjects suffering from thyroid cancer and melanomas. Haematological cancers include leukaemia, lymphoma and myeloma. The immunocompromised condition of the individual may be due to a cancer disease as mentioned herein or the treatment of said cancer disease.

### BACKGROUND OF THE INVENTION

Solid Tumors

A solid tumor is a cancer of a body tissue other than blood, bone marrow, or the lymphatic system. Examples of solid tumors include breast cancer, prostate cancer, and cancers of the lungs, bladder, kidneys, colon and thyroid. The cancers that present with solid tumors can be divided into several groups, referred to as female cancers, male cancers, cancers of the respiratory system, cancers of the gastro intestinal system, cancers of the renal system and further thyroid cancer and melanomas are described.

Treatment

The treatment of any of the above mentioned cancer disease, may involve surgery, chemotherapy and/or radiation therapy.

Depending on the cancer treated, the surgery may be small or large. Especially for major surgeries, which are often employed for the treatment of cancers, such as gastric cancers, the recovery may be very involved. The patient may experience post-surgical pain, weakness, fatigue, loss of appetite and other effects. Surgical procedures may further cause lymphocytopenia—low number of lymphocytes (white blood cells) in the blood.

Chemotherapy is a systemic treatment that uses drugs to destroy cancer cells. It is most often administered orally or intravenously although local treatment may be employed for specific disease. Chemotherapy also affects healthy dividing cell, such as the lining of the digestive tract, the hair roots, and blood cells. Therefore side effects of chemotherapy used in the treatment of cancer include, nausea, vomiting, weakness, loss of appetite (anorexia), fatigue, headache, increased risk for infection, loss of hair (alopecia), leucopenia, Neutropenia and anemia. Anemia is characterised by; a weakening of the ability of the red blood cells to carry oxygen, a reduced blood clotting ability and a reduced immune response which may be associated with the experienced fatigue and weakness. Neutropenia is characterized with an increased risk of infections due to reduced phagocytic function by the lack of neutrophils and monocytes. Further side-effects of treatment of for example gastric cancer, bladder cancer and urethral cancer may includes diarrhea, mouth sores, abdominal pain, bladder irritation, blurred vision, shortness of breath, excessive bleeding and bruising,

Radiation uses high-energy x-rays to destroy cancer cells and to relieve symptoms (called palliative treatment). Side effects of radiation therapy depend on the local areas affected and often include skin irritation, fibrosis, diarrhea, fatigue and nausea. Treatment of for example bladder cancer may have the side-effects, such as inflammation of the rectum (proctitis), incontinence, hematuria (blood in the urine), fibrosis, and impotence, whereas the most common side effects of treating urethral cancer is: abnormal healing resulting in abnormal passage in the urethra (fistula), burning of the skin (similar to sunburn), inflammation of the bladder (cystitis) and narrowing of the urethra (stricture; causing urination difficulty).

Thus the above mentioned treatments, surgery, chemotherapy and radiation therapy have a severe impact on the body and are often accompanied by inhibition of the immune system and the phagocytic cells in the individual, whereby the patient may have an increase susceptibility to infections by micro-organisms, such as bacterial, viral and fungal infections. Such infection may have severe impact on people already weakened by a cancer disease, thus prevention or treatment of infections in cancer patients, may also improve the recovery from the cancer and thereby be a treatment of cancers.

MBL

Several groups of lectins, i.e., carbohydrate-binding proteins, are known in man. One group is the C-type lectins. The C-type lectins contain a calcium-dependent carbohydrate recognition domain (a C-type CRD)¹. Mannan-binding lectin (MBL), synonymous to mannose-binding lectin, mannan-binding protein or mannose-binding protein (MBP), belongs to the subgroup of C-type lectins, termed collecting, since these soluble proteins are composed of subunits presenting three CRDs attached to a collagenous stalk². MBL interact with carbohydrates presented by a wide range of micro-organisms and accumulating evidence shows that it plays an important role in the innate immune defence³. When bound to carbohydrate MBL is able to activate the complement system.

The complement system may be activated via three different pathways: the classical pathway, the alternative pathway, and the newly described third pathway, the mannan-binding lectin (MBL) pathway which is initiated by the binding of MBL to carbohydrates presented by micro-organisms. The components of the alternative pathway and of the MBL pathway are parts of the innate immune defence, also termed the natural or the non-clonal, immune defence, while the classical pathway involves co-operation with antibodies of the specific immune defence⁴.

The human MBL protein is composed of up to 18 identical 32 kDa polypeptide chains²⁷, each comprising a short N-terminal segment of 21 amino acids including three cysteine residues, followed by 7 repeats of the collagenous motif Gly-X—Y interrupted by a Gln residues followed by another 12 Gly-X—Y repeats. A small 34 residue ‘neck-region’ joins the C-terminal Ca²⁺-dependent lectin domain of 93 amino acids with the collagenous part of the molecule²⁸.

The collagenous regions of the three polypeptide chains combine to form a subunit which is stabilised covalently by disulphide bridges. Individual subunits are joined by disulphide bridges as well as by non-covalently interactions²⁷.

The position of these disulphide bridges has, however, not been fully resolved. SDS-PAGE analysis under non-reducing conditions of MBL shows bands with an apparent molecular weight (m.w.) larger than 200 kDa presumably representing blocks of 3, 4, 5 and even 6 assembled subunits²⁷.

The actual number of subunits in the natural human MBL protein has been controversial. Lipscombe et al. (1995) obtained data by use of ultracentrifugation suggesting 25% of human serum MBL to be made of 2-3 subunits and only a minor fraction reaching the size of 6 subunits. The relative quantification was carried out by densitometry of Western blots developed by chemiluminescence²⁷ found by SDS-PAGE analysis of fractions from ion exchange chromatography that the predominant species of covalently linked MBL subunit chains consisted of tetramers while only pentameric or hexameric complexes activated complement. Gel permeation chromatography (GPC) analysis, in contrast, suggests that MBL is comparable in size with the C1 complex. GPC can be carried out under conditions which allow for a study of the importance of weak protein-protein interactions in the formation of MBL molecules. MBL content in the GPC fractions can be determined by standard MBL assay techniques.

MBL is synthesized in the liver by hepatocytes and secreted into the blood. It binds to carbohydrate structures on bacteria, yeast, parasitic protozoa and viruses, and exhibits antibacterial activity through killing of the microorganisms by activation of the terminal, lytic complement components or through promotion of phagocytosis (opsonization). The sertiform structure of MBL is quite similar to the bouquet-like structure of C1q, the immunoglobulin-binding subcomponent of the first component in the classical pathway³. C1q is associated with two serine proteases, C1r and C1s, to form the C1 complex. Similarly, MBL is associated with two serine proteases MASP-1⁵ and MASP-2⁶, and an additional protein called Map19⁷. MASP-1 and MASP-2 have modular structures identical to those of C1r and C1s⁶. The binding of MBL to carbohydrates induces the activation of MASP-1 and MASP-2. MASP-2 then generates the C3 convertase, C4bC2b, through cleavage of C4 and C2. Reports suggest that MASP-1 may activate C3 directly. Nothing is known about the stoichiometry and activation sequence of the MBL/MASP complexes. MBL has also been characterized in other animals such as rodents, cattle, chicken and monkeys.

The concentration of MBL in human serum is largely genetically determined, but reportedly increases up to threefold during acute phase reactions⁸. Three mutations causing structural alterations and two mutations in the promoter region are associated with MBL deficiency⁹. MBL deficiency is associated with susceptibility to a variety of infections. Examination of five adult individuals with unusual and severe infections showed three to be homozygous for structural MBL mutations and two to be heterozygous¹⁰. Investigation of 229 children referred to the Danish National Hospital because of non-HIV-related immunodeficiency showed a tenfold higher frequency of homozygosity for structural MBL mutant alleles than seen in a control group¹¹. Allotyping of 617 consecutively hospitalized children at St Mary's Hospital in London showed significantly higher frequency of homozygosity and heterozygosity for mutant allotypes in the infected children than in the noninfected¹².

A wide range of oligosaccharides can bind to MBL. As the target sugars are not normally exposed on mammalian cell surfaces at high densities, MBL does not usually recognize self-determinants, but is particularly well suited to interactions with microbial cell surfaces presenting repetitive carbohydrate determinants. In vitro, yeast (Candida albicans and Cryptococcus neoformans), viruses (HIV-1, HIV-2, HSV-2, and various types of influenza A) and a number of bacteria have been shown to be recognized by MBL. In the case of some bacteria, the binding with MBL is impaired by the presence of a capsule¹³. However, even encapsulated bacteria (Neisseria meningitidis) can show strong binding of MBL¹⁴.

The microorganisms which infect MBL deficient individuals, represent many different species of bacterial, viral and fungal origin^{12, 15-17}. Deficiency is also associated with habitual abortions¹⁸. Indeed, MBL could be a general defence molecule against most bacteria, and thus be considered as one reason why so many bacteria are non-pathogenic.

While accumulating data support the notion of a protective effect of MBL there are also observations suggesting that infections with some microorganisms, notably intracellular pathogens, attain a higher frequency in MBL sufficient than in MBL deficient individuals^{19, 20}. This is in concordance with the results of an animal experiment, where an increased number of HSV-2 were found in the liver of mice pre injected with human MBL²¹.

Clinical grade MBL has been obtained from blood donor plasma and shown to be safe upon infusion²². Production of recombinant MBL conceivably having a structure and an activity similar to that of native MBL has been attained (patent application PA 1999 00668/C5/KH).

### SUMMARY OF THE INVENTION

The invention features the use of MBL, purified from natural sources or from material produced by recombinant technologies, or by any other suitable MBL-producing cell line, for the prophylaxis and/or treatment of infections in an individual having a solid tumor or haematological cancer and/or having received medical treatment for such cancer disease. Thus the infections disease may be associated with a therapeutic or medical treatment, such as e.g. surgery and the use of cytotoxic therapy. The MBL may be given before or after start of the medical treatment and for any duration of time deemed suitable.

The invention also relates to treating individuals having normal MBL levels, as such individuals are likely to benefit—prior to and/or after the start of any surgery or cell toxic treatment—from MBL administration.

Accordingly, the invention in one aspect relates to treatment and/or prophylaxis of infections in individuals suffering from an immunocompromising condition, or to treatment of individuals who are likely be immunocompromised due to medical treatment known to be associated with the occurrence of an immunocompromised condition.

Medical treatment of many types of cancer frequently involves surgery as describe above, whereby the diseased tissue is removed. Rarely surgery is the only treatment as surgery is combined with chemotherapy and radiation therapy such as e.g. x-ray treatment or the like for obtaining better results.

Surgery may be considered an immunosuppressant treatment as the post operative symptoms include pain, weakness, loss of appetite and anaemia where by the strength of the immune response in a subject is weakened and therefore the subject is more susceptible to infections.

Chemotherapy and radiation therapy are offered as part of the treatment of several forms of cancers, aiming either at slowing the progression of the disease or reversing said progression by means of a curative treatment. Chemotherapy and radiation therapy are immunocompromising therapy since cells of the immune system may be killed, thus leading to a state of immunosuppression especially characterized by neutropenia. Cytotoxic treatment may also affect the immune system indirectly as tissue damages through out the body may prevent the immune system from responding normally to infections agents and thereby leaving the patient more susceptible to microbial species.

MBL is believed to exert its antimicrobial activity mainly through its opsonizing activity (preparation of microorganisms for phagocytosis). This activity is dependent on activation of complement after binding of MBL to the microbial surface and deposition of C4b and C3b on the microorganism. MBL can also promote the direct complement-mediated killing of the microorganism through the activation of the terminal lytic pathway of complement and insertion of the membrane attack complex (MAC) in the membrane. This mechanism is considered of minor importance. Many microorganisms, such as Gram-positive bacteria, e.g., Streptococcus pneumonia, are resistant to MAC, but can be eliminated by opsonophagocytosis. Considering opsonophagocytosis as the main effector mechanism of MBL-mediated clearance of microorganisms, it is a surprise that MBL treatment could be of benefit to persons being deficient of the most important phagocytic cells, i.e., the neutrophiles.

The importance of neutropenia in the risk of serious infections in individuals with cancer who are receiving cytotoxic chemotherapy was recognized nearly 30 years ago²³. Infections thus occur very frequent in cancer patients undergoing chemotherapy and other immunocompromising therapeutic interventions. Synchronous with the intensified use of chemotherapy, problems with infections are increasing and are now a major challenge in supportive care²⁴. Accordingly, all oncology departments use many resources on fighting infections. This fight is steadily getting more difficult due to the appearance of multi resistant bacterial strains.

Individuals being devoid of the important cellular components of the immune system having an immunocompromising condition or receiving an immunocompromising treatment are dependent on an efficient innate humoral immune system, such as the complement system. This includes individuals experiencing periods of neutropenia. No sufficiently accurate and reliable prognostic factors are presently capable of predicting an increased risk of serious infections in individuals treated with chemotherapy, radiation therapy, or other immunocompromising treatments²⁴. Accordingly, an immunocompromising condition arising from a medical treatment is likely to expose the individual in question to a higher risk of infection. It is possible according to the invention to prophylactically treat an infection in an individual having the immunocompromising condition before or during treatments known to generate such a condition. By prophylactically treating with MBL before or during a treatment known to generate such a condition it is possible to prevent a subsequent infection or to reduce the risk of the individual contracting an infection due to the immunocompromising condition. Should the individual contract an infection e.g. following a treatment leading to an immunocompromised condition it is also possible to treat the infection by administering to the individual an MBL composition according to the invention.

The invention is also directed to treatments of such deficiencies by infusion of MBL. Furthermore, the invention is directed to the use of MBL plasma concentrations for predicting the risk of infection of individuals undergoing e.g. chemotherapy.

In another aspect the present invention is related to the use of a composition comprising at least one mannan-binding lectin (MBL) subunit, or at least one oligomer comprising the at least one mannan-binding lectin (MBL) subunit, in the manufacture of a medicament for prophylactic, ameliorating or curative treatment of an infection in an individual having an immunocompromised condition caused by a solid tumor or a haematological cancer or/and the treatment of said tumor or cancer and/or an individual being at risk of acquiring an immunocompromised condition resulting from a solid tumor or a haematological cancer or/and a medical treatment of said tumor or cancer.

In an embodiment the individual initially having plasma levels of MBL in excess of 50 ng/ml. In particular the individual may be genetically disposed to an MBL deficiency or have acquired an MBL deficiency leading to an increased risk of suffering from infections. Accordingly, the invention also concerns treatment of infections in individuals suffering from a mannan-binding lectin (MBL) deficiency including any deficiency in the production of MBL and/or function of MBL.

In yet another aspect there is provided a method for estimating the probability of the occurrence of any clinically significant infection in an individual being immunocompriomised by a solid tumor or a haematological cancer or the treatment of said cancer disease, said method comprising the step of measuring the concentration of MBL in plasma or serum obtained from the individual, and estimating the probability on the basis of the measured concentration.

In the present context immunocompromised is used in its normal meaning, i.e. an individual not being capable of evoking an adequate immune response due to primary or secondary deficiency, induced or non-induced, in one or more of the elements of the normal immune defence system.

### DETAILED DESCRIPTION OF THE INVENTION

The present invention describes that treatment of solid tumors or haematological cancers may be improved by MBL treatment and further that concurrent infections in patients suffering from such cancers may be prevented by MBL treatment.

In the following, several groups of solid cancers, referred to as female cancers, male cancers, cancers of the respiratory system, cancers of the gastro intestinal system, cancers of the renal system and further thyroid cancer and melanomas are described.

Female Cancers

The most common and serious female specific malignancies occur in the breasts, ovaries, endometrium (lining of the uterus), cervix, vulva, and vagina. These sites of origin are often considered as a single group, although the ethology underlying the disease may be different. Female cancers including, breast cancer, cervical cancer, uterus cancer and ovarian cancer are all serious disease that are currently treated by surgery, radiation therapy and/or chemotherapy. As the treatment methods are not sufficiently effective several experimental treatments are also used. The risk of developing female cancers may increase in females; who take estrogen, are post-menopausal, are obese, or have high blood pressure. As many female cancers are inheritable regular screenings may be used in individual with an increased risk of developing cancer, whereby the disease may be detected at an early and more treatable stage.

Male Cancers

The most common male cancers are prostate cancer and testicular cancer. The prostate is a gland secreting the seminal fluid. Prostate cancer occurs when cells grow abnormally in the prostate gland to form tumors. Prostate gland tumor growth can be fuelled by the male sex hormone, testosterone. The testicles are part of the male reproductive system and produce several male hormones, including testosterone. The hormones control the development of the reproductive organs, as well as other male characteristics. Testicular cancer is one of the most curable forms of cancer. When testicular cancer spreads, the cancer cells are carried by blood or by lymph. The exact cause of testicular cancer is unknown. Possible risk factors include the following: age (prevalent cancer between the age of 15 to 40), undescended testicle(s), Klinefelter's syndrome, family history, personal history of cancer in the other testicle, race and ethnicity (the rate of testicular cancer is higher in Caucasians than in other populations) and HIV infection although many men with testicular cancer do not have the suggested risk factors. The male cancers are presently some of the most curable cancers if diagnosed at an early time point. The treatment methods employed depends on the individual and the stage of the disease. Radical inguinal orchiectomy surgery is used to remove the tumor and the testicle, external radiation may be used, as well as chemotherapy and even high-dose chemotherapy followed by stem cell transplantation may be used to help the patient to produce healthy blood cells.

Cancers of the Respiratory System

The tissue of the respiratory system is greatly affected by the air that we breathe. Examples of cancers of the respiratory system include nasopharyngeal cancer and lung cancer as described here below.

Nasopharyngeal Cancer

The nasopharynx is the upper part of the throat behind the nose—the nostrils lead into it, and openings on the sides of the nasopharynx connect to the ears. Many factors may influence the development of nasopharyngeal cancer, including diet (nitrosamines released when cooking salt-cured meats and fish may be distribute over the mucous membranes of the nasopharynx (nasopharyngeal mucosa), viruses such as Epstein-Barr virus (EBV) and genetic susceptibility. The disease has also been associated with certain other cancers, including Burkitt's lymphoma, immunoblastic lymphoma. Radiation therapy is the standard treatment for almost all Nasopharyngeal cancers. Due to the high likelihood of lymph node involvement, treatment usually includes radiation therapy to both sides of the neck. In advanced forms of the disease, radiation alone is rarely sufficient to control tumor growth. Patients with advanced tumors typically are treated with a combination of chemotherapy and radiation, followed by adjuvant chemotherapy—additional drug treatment for patients whose cancers are thought to have spread outside their original sites.

Lung Cancer

The lungs are the body's major organs of respiration. The two vital parts that make up the lungs are located on each side of the chest within the rib cage. The lining of the tracheobronchial tree of the lungs is composed of columnar epithelium (column-shaped surface cells) and glands that produce mucus and serous fluid (clear plasma). The cilia (hair-like projections on columnar epithelium) move in a constant, beating motion to cleanse the airways of foreign bodies and infectious organisms. Lungs that have been damaged by smoking or other toxic exposures often have defective or missing cilia and show other abnormalities in the tissue lining. The main risk factors of lung cancer are smoking and additional risk factors include such as secondhand smoke, asbestos, radon, occupational exposures, age, race, sex, and heredity may also play a role in lung cancer development. Additionally, there are some studies suggesting that lung cancer may influenced by the diet as dietary micronutrients such as carotenoids, vitamin C, vitamin E, and selenium appear to have a positive effect.

Lung cancers are often divided into two groups: small cell lung carcinoma (SCLC) and non-small cell lung carcinoma (NSCLC) where the latter include squamous cell carcinoma, adenocarcinoma, and large cell carcinoma. Large cell carcinoma and adenocarcinoma usually are found on the periphery (outer edges) of the lungs and may occur as solitary nodules, masses, or scar cancer. By contrast, squamous cell carcinoma and small cell carcinoma often are centrally located and may appear to be pneumonia (inflammation of the lungs), atelectasis (collapsed lung), or pit-like masses.

Surgical resection or cutting away, of the tumor is generally the employed treatment methods for disease that has not spread beyond the lung, except for the treatment of small cell lung cancer where a systemic approach that includes chemotherapy and local control with radiotherapy is employed. If the tumor is more aggressive and/or widespread, chemotherapy and radiotherapy (radiation therapy) may also be necessary. Unfortunately, surgical procedures may cause lymphocytopenia—low number of lymphocytes (white blood cells) in the blood—which is linked with shorter survival times among patients with advanced lung cancer. Although lung cancer survival rates have improved the rate of survival continues to be low in comparison to other cancers.

Cancers of the Gastrointestinal System

Organs as the stomach, liver, pancreas, colon, and spleen are closely located in the cavity of the abdomen. Also the kidneys and adrenal glands are similarly located, and cancers affecting one of these organs may easily involve one or more of the organs present in the vicinity. Thus the cancers included are gastric cancer, pancreatic cancer, colorectal cancer and hepatic cancer. Also peritoneal cancers may be included.

Gastric Cancer

The major functions of the stomach are related to digestion of food. After swallowing, the food is held in the stomach temporarily. The mucus lining of the stomach secretes acidic gastric juices. These act on the food and begin to break it down chemically. The periodical contractions of the stomach, combined with the opening and closing of the pyloric valve, move the partially digested food into the duodenum. There are a variety of risk factors associated with gastric cancer, such as age, race, diet (gastric cancer may be associated with high intakes of dried and/or salted foods), medical factors, as gastric cancer is more common among individuals with histories of the following conditions: pernicious anemia, atrophic gastritis (Menetrier's disease), intestinal polyps (non-cancerous mushroom-shaped growths), previous gastric cancer and infection by Heliobacter pylori (this species of bacteria is related to stomach ulcers, yet ulcers themselves do not seem to be associated with gastric cancer) and smoking.

Pancreatic Cancer

The pancreas is located near the stomach and the small intestine. The two major functions of the pancreas is production of enzymes that help in the digestion of food products (exocrine function) and production of several hormones that have diverse functions (endocrine function). Cancers of the pancreas can occur in the exocrine pancreas (classic pancreatic adenocarcinomas) or in the endocrine pancreas. Risk factors for the development pancreatic cancer include smoking, diet, environmental factors such as long-term exposure to certain chemicals, such as gasoline and related compounds, as well as certain insecticides, medical/surgical factors as a histories of the following conditions: cirrhosis (a chronic liver disease), chronic pancreatitis, diabetes or surgery to the upper digestive tract and genetic factors. Due to difficulties in diagnosis, the intrinsic aggressive nature of pancreatic cancers, and the sparse systemic treatment options available pancreatic cancer have low survival rate.

Colorectal Cancer

The gastro-intestinal tract may be affected by malignancies in several places. The colon and rectum are located in the abdomen between the small intestine and the anus. Together, the colon and rectum make up the large intestine or large bowel. The appendix is a small outpouching from the beginning of the large intestine (the ascending colon). Cancer of the gastro intestinal tract such as bowel cancer, colon cancer, rectal cancer, anal cancer and appendix cancers are therefore closely related.

The risk factors of colorectal cancer includes; age, diet, ethnic group, sedentary lifestyle, genetic predisposition and medical factors are more common among individuals with histories of the following conditions: intestinal polyps (noncancerous mushroom-shaped growths), chronic inflammatory bowel disease (ulcerative colitis or Crohn's colitis), and previous colorectal cancer. Women who have had cancers of the breast, uterus or ovary may also have a higher risk of developing colorectal cancer.

Hepatobiliary Tumors

Cancers of the liver, bile ducts, and biliary tract, the tubes that carry bile from the liver or gallbladder to the small intestine are collectively referred to as hepatobiliary tumors and include hepatocellular carcinoma (primary liver cancer). The liver is located on the right side of the abdomen and is protected by the rib cage. The liver is essential for life and perform many functions, such as cleaning of the blood, conversion of food into energy, manufactures essential body proteins that transport substances in the blood associated with proper blood clotting and resistance to infection, produces bile, which eliminates toxic substances from the body and aids digestion, helps to regulates the balance of sex hormones, thyroid hormones, cortisone and other adrenal hormones, produces, excretes, and converts body cholesterol to other essential substances, regulates the body's supply of essential vitamins and minerals, such as iron and copper. Hepatocarcinogens are naturally occurring substances. The best studied and most potent of these agents appears to be aflatoxin B1, produced by the fungus aspergillus, which can be found in a variety of stored grains. Other naturally occurring agents include senecio plants and the cycad plants. Other risk factors include medical conditions as cirrhosis and metabolic diseases. Cirrhosis induced by a variety of conditions, such as viral infections of the liver, principally hepatitis B and hepatitis C; excessive alcohol consumption; autoimmune chronic active hepatitis; and cryptogenic cirrhosis (cirrhosis with obscure or unknown origin). Metabolic diseases may be such as hemochromatosis (accumulation of iron in the liver and other organs); glycogen storage diseases; Wilson's disease (accumulation of copper in the body); and galactosemia (accumulation of galactose in the blood). Risk factors further include such as excessive exposure to; thorotrast (a contrast agent once used in x-ray exams but now discontinued), androgenic steroids and polyvinylchloride.

The treatment of choice for cancers of the gastro intestinal system such as gastric cancer, pancreatic cancer, colorectal cancer and hepatic cancer is usually surgery. Other possible treatments include chemotherapy, radiation and immunotherapy and combinations thereof. In many cases, the surgery for, for example gastric cancer will require a large incision in the abdomen.

Cancers of the Renal System

The kidneys together with the urinary tract (renal pelvis, ureters, bladder, urethra) form the renal system. The kidneys are composed of about one million microscopic “filtering packets” called glomeruli. The glomeruli remove uremic waste products from the blood. Urine made by the glomerulus moves down the tubule. Together, the glomerulus and the tubule form a unit called a nephron. Each nephron connects to progressively larger tubular branches, until it reaches a large collection area called the calyx. The calices form the funnel-shaped portion of the upper ureter (renal pelvis). Urine moves from the renal pelvis through the ureters, before reaching the bladder and finally being excreted from the body via the urethra. Apart from filtering the urine the kidneys produce three important hormones: erythropoietin (EPO), renin, and vitamin D. Risk factors of cancers of the renal system include; smoking, exposure to carcinogens in the urine, personal history of bladder cancer, chronic bladder inflammation (recurrent urinary tract infections, urinary stones), infection with Schistosoma haematobium (parasite found in many developing countries), consumption of Aristolochia fangchi (herb used in some weight-loss formulas), family history of bladder cancer, treatment with or occupational exposure to certain drugs (antineoplastic drugs, e.g. cyclophosfamide and dyes containing aniline), age, gender, race and diet.

Several types of cancer can develop in the renal system. Renal cell carcinoma (RCC) is the most common cancer form of the kidneys. In RCC, cancerous (malignant) cells develop in the lining of the kidney's tubules. In most cases, a single tumor develops, although more than one tumor can develop within one or both kidneys. Bladder cancer occurs frequently whereas urethral cancer is rare and is often associated with invasive bladder cancer.

Treatment for cancer of the renal system depends on the stage of the disease, the type of cancer, and the patient's age and overall health. Options include surgery, chemotherapy, radiation, hormone therapy and immunotherapy. In some cases, treatments are combined. For the treatment of early bladder cancer, chemotherapy may be infused into the bladder through the urethra (called intravesical chemotherapy). Drugs commonly used to treat bladder cancer include valrubicin (Valstar™), thiotepa (Thioplex®), mitomycin, and doxorubicin (Rubex®). In the treatment of urethral cancer cisplatin (Platinol®), vincristine (Oncovin®), and methotrexate (Trexall®) are commonly used.

Tumors of the Endocrine System

The adrenal glands, the parathyroid glands and the thyroid are also subject of cancer diseases. Adenomas of the parathyroid gland, adrenal tumors and thyroid cancer are examples of cancers of the endocrine system.

Thyroid Cancer

The thyroid is a butterfly-shaped gland located in the neck, below the Adam's apple. Hormones produced by the thyroid regulate heart rate, blood pressure, body temperature, metabolism, and affects the nervous system, muscles, and other organs. Thus thyroid cancer may have severe affects on many body functions. There are four major types of thyroid cancer: papillary, follicular, medullary, and anaplastic. Papillary cancer de develops in the hormone producing cells. Papillary cancer grows very slowly and contains cells that are similar to healthy thyroid cells. The treatment of papillary cancer is often successful. Follicular cancer also develops in the hormone producing cells. Many follicular cancers can be cured although the disease may be difficult to control if the cancer invades blood vessels or grows into nearby structures in the neck. The cells involved in medullary cancers produce calcitonin. Medullary cancer is more difficult to control than papillary and follicular thyroid cancers because the cancer cells tend to spread to other parts of the body. Anaplastic cancer is the fastest growing type of thyroid cancer. The cancer cells are extremely abnormal and spread rapidly to other parts of the body. Of the four types of thyroid cancer, only medullary thyroid cancer has a clear genetic predisposition. This predisposition is due to an alteration of the RET gene. Individuals who inherit this alteration are almost certain to develop medullary thyroid cancer at some time in their lives.

The risk factors of thyroid cancer include age, gender, race, x-ray therapy, radioactive products, including radioactive iodine (mainly I¹³¹).

Surgery is the main treatment for most types of thyroid cancer, but other therapies including Radioiodine therapy and radiation, may be use depending on the type of thyroid cancer you have. Active iodine therapy may serve two functions, both to destroy any normal tissue that remains after surgery and to destroy any cancer that has spread beyond the thyroid gland. Radioiodine therapy is a standard treatment for follicular cancer and may sometimes be used in people with papillary cancer.

Melanoma

Melanoma develops most of in the cells of you skin that produce melanin (melanocytes). It can also form in the eyes (intraocular melanoma) and in rare cases in internal organs such as the intestine. Melanomas are more likely than other skin cancers to metastasize, and are thus more difficult to treat. The risk factors of melanoma include exposure to ultraviolet (UV) radiation (sunlight or tanning lamps), age, carcinogens and heredity. Melanoma can be successfully treated if it's caught at an early stage. The standard treatment for melanomas that haven't spread beyond the skin is surgery to remove the cancer either by a simple excision of a wide local excision in case of a more invasive tumor. When melanoma has spread to another part of the body, options may include surgery, chemotherapy, radiation therapy, biological therapy, experimental therapy or a combination.

Haematological Cancer

The haematological cancers—leukaemia, lymphoma and myeloma—are cancers arising from abnormal blood or bone marrow cells.

Leukemia

There are four main types of leukaemia: acute lymphoblastic leukaemia (ALL), acute myeloid leukaemia (AML), chronic lymphocytic leukaemia (CLL), chronic myeloid leukaemia (CML) and a fifth less common leukaemia; hairy cell leukaemia. The chronic diseases develop very slowly whereas the acute disease develops fast.

In myeloid cancers too many granulocytes (blast cells) are produced and released into the blood although immature and unable to work properly. The production of other types of blood cells is also disrupted, such as the white blood cells that divide and mature abnormally.

Lymphocytic cancer is an overproduction of immature lymphocytes that fill up the bone marrow and prevent it from making blood cells properly. The lymphocytes multiply too quickly and live too long, so there are too many of them circulating in the blood. They are not fully developed and do not work properly. Over a period of time the abnormal cells replace the normal white cells, red cells and platelets in the bone marrow whereby the production of red cells and platelets is affected.

Lymphoma

There are two main types of lymphoma. One is called Hodgkin's disease the other is called non-Hodgkin's lymphoma. There are about 20 different types of non-Hodgkin's lymphoma including; anaplastic large cell, follicular, small lymphocytic, lymphoblastic, extranodal marginal zone (MALT), mantle cell, primary mediastinal large, splenic marginal zone, nodal marginal zone, primary effusion, B-cell prolymphocytic, precursor B lymphoblastic, nodal marginal zone B-cell, diffuse large B-cell, peripheral T-cell, cutaneous T cell, Waldenstroms macroglobulinaemia (lymphoplasmacytic) and Burkitt's.

Myeloma

Myeloma, also known as multiple myeloma or myelomatosis, is a cancer of plasma cells. The cancer cells fill up the bone marrow and interfere with production of the normal white cells, red cells and platelets. The myeloma cells usually produce a large amount of one type of abnormal antibody termed paraprotein or M protein. Thus myeloma interferes with the immune system. The myeloma cells have the ability to spread throughout the bone marrow and into the hard outer casing of the bone in many parts of the body. Myeloma is rarely curable and treatment is usually able only to control it, leading to an improvement in symptoms and a better quality of life. Chemotherapy, usually combined with steroids, is the main treatment for myeloma. Stem cell transplant may also be useful. Radiotherapy can strengthen the bone and reduce pain in the affected areas. Surgery may also occasionally be used to strengthen weakened bones, to prevent fractures or, rarely, remove areas of myeloma that are pressing on important areas such as the spinal cord. Other treatments may be needed, such as pain killers to treat bone pain, blood transfusions if you are anaemic, or kidney dialysis if the kidneys are failing.

MBL

Until now MBL has been used for treating MBL deficiency as such which has been defined by an arbitrary level of below 50 ng/ml, or more often below 10 ng/ml serum which is often identical with the sensitivity of various MBL test assays, and the level has therefore been set as the level for which substantially no MBL could be detected in the various prior art assays.

By the present invention it is described infections may be prevention and/or treated in immunocompromised individuals where the subject is immunocompromised or expected to become immunocompromised due to treatment of a disease such as the medical treatment of variety of solid tumors or haematological cancers.

The medical treatment of solid tumors or haematological cancers is often accompanied by inhibition of the immune system in the individual, whereby the patient may have an increase susceptibility to infections by micro-organisms, such as bacterial, viral and fungal infections. Such infection may have severe impact on people already weakened by a cancer disease, thus prevention or treatment of infections in cancer patients, may also improve the recovery from the cancer and thereby be a treatment of cancers. Chronic or recurrent infection are also proposed as being risk factors of several cancers, thus MBL treatment may be considered prophylactic treatment to prevent development of cancer.

Thus MBL, may be used as prophylactic or curative treatment before, simultaneously or after other treatments of solid tumors, such as female cancers, male cancers, cancers of the respiratory system, cancers of the gastro intestinal system, the renal system and further thyroid cancer and melanomas.

Thus MBL, may further be used as prophylactic or curative treatment before, simultaneously or after other treatments of haematological cancers, such as leukaemia, lymphoma and myeloma, wherein the leukaemia may be acute lymphoblastic leukaemia (ALL), acute myeloid leukaemia (AML), chronic lymphocytic leukaemia (CLL), chronic myeloid leukaemia (CML) or hairy cell leukaemia and wherein lymphoma may be Hodgkin's disease or non-Hodgkin's lymphoma.

Such treatment is independent on their serum MBL level of the immunocomprised individual. In particular infections may be prevented in immunocompromised individuals when administering MBL to these individuals having an MBL level in excess of 50 ng/ml serum. Also, individuals having an MBL level in excess of 75 ng/ml serum may be in need of treatment, such as individuals having an MBL level in excess of 100 ng/ml serum, and individuals having an MBL level in excess of 150 ng/ml serum.

Also the MBL treatment or prevention of infections may be conducted by administering MBL to these individuals in combination with relevant antibiotics, anti-viral agents or anti-fungal agents.

In particular, individuals at risk of acquiring an immunocompromised condition resulting from a medical treatment will benefit from being prophylactically treated with MBL before, during and maybe also after the treatment in order to prevent diseases associated with the immunocompromised condition, such as infections.

Generally all individuals being immunocompromised or at risk of becoming immunocompromised should be treated with MBL independent on their specific MBL level. The reason behind this is that infection may lead to MBL depletion, and therefore an MBL “booster”, increasing the MBL level initially will reduce the risk of MBL depletion to a level below a deficiency level, and the immune defence of these patients can be reinforced by administration of recombinant or natural plasma-derived MBL. In particular infections may be prevented when administering MBL to individuals having an MBL level in excess of 50 ng/ml serum. Also, individuals having an MBL level in excess of 75 ng/ml serum may be in need of treatment, such as individuals having an MBL level in excess of 100 ng/ml serum, and individuals having an MBL level in excess of 150 ng/ml serum.

The present inventors have also shown herein that in particular individuals having an MBL level below 500 ng/ml serum will benefit from MBL treatment in relation to an immunocompromised condition. Consequently, in particular individuals having an MBL level below 400 ng/ml will benefit, such as individuals having an MBL level below 300 ng/ml, such as individuals having an MBL level below 250 ng/ml, such as individuals having an MBL level below 200 ng/ml.

Thus, in a preferred embodiment the present invention relates to the use of MBL for manufacturing of a medicament for of individuals having an MBL level in serum in the range of 50-500 ng/ml, such as in the range of 100-500 ng/ml for treating and/or preventing infections, in particular in relation to an immunocompromised condition of the individual.

The immunocompromised condition may be due to a medical treatment as discussed above, i.e. chemotherapy, radiation therapy or other immunosuppressing treatment, such as induced by treatment with steroids, cyclophosphamide, azathioprine, metotrexate, cyclosporine, and/or rapamycin, or other drugs used in cancer treatment.

Also, surgery may be considered an immunosuppressing treatment as the patient during convalescence is weakened and thus have an increased risk of acquiring infections.

Furthermore, individuals having an MBL level above 50 ng/ml and below 500 ng/ml will benefit from MBL treatment in general, in order to prevent infections, in particular chronic infections.

One group of individuals being in need of MBL treatment in order to prevent and/or treat infections are individuals having a low level of functional MBL, independent on the level of MBL as such. This is due to the fact, that for some mutations of the MBL it has been found, that although MBL subunits and oligomers thereof are expressed in serum the functionality thereof are low. The functionality or functional activity of MBL may be estimated by its capacity to form an MBL/MASP complex leading to activation of the complement system. When C4 is cleaved by MBL/MASP an active thiol-ester is exposed and C4 becomes covalently attached to nearby nucleophilic groups. A substantial part of the C4b will thus become attached to the coated plastic well and may be detected by anti-C4 antibody.

A quantitative TRIFMA for MBL functional activity is constructed by 1) coating micro-titre wells with 1 mg mannan in 100 ml buffer; 2) blocking with Tween-20; 3) applying test samples, e.g. diluted MBL preparations 4) applying MBL deficient serum (this leads to the formation of the MBL/MASP complex); alternatively the MBL and the MBL deficient serum may be mixed before application with the microtitre wells; 5) applying purified complement factor C4 at 5 mg/ml; 6) incubate for one hour at 37° C.; 7) applying Eu-labelled anti-C4 antibody; 8) applying enhancement solution; and 9) reading the Eu by time resolved fluorometry. Between each step the plate is incubated at room temperature and washed, except between step 8 and 9.

Estimation by ELISA may be carried out similarly, e.g. by applying biotin-labelled anti-C4 in step 7; 8) apply alkaline phosphatase-labelled avidin; 9) apply substrate; and 10) read the colour intensity.

The functionality may be expressed as the specific activity of MBL, such as 1 unit of MBL activity per ng MBL. A non-functional MBL may be defined as MBL having a specific activity less than 50% of plasma MBL specific activity, such as less than 25% of plasma MBL specific activity, wherein the plasma MBL is purified from an individual not suffering from any MBL mutations. In particular the reference plasma MBL is plasma pool LJ 6.57 28/04/97.

Thus, the present invention also relates to the prevention and/or treatment of infection in individuals having a mutation in their MBL gene leading to a reduced expression of MBL and/or expression of non-functional MBL.

In particular such mutations in the MBL gene can lead to a change of aminoacid number 52 (numbering including the leader peptide of MBL) from arginine to cysteine, aminoacid number 54 from glycine to aspartic acid or amino acid number 75 from glycine to glutamic acid.

Also mutations in the promoter region of the MBL gene can lead to lowered levels of MBL. In particular mutations at position −221 have an influence on the expression of MBL.

The MBL sequence may be found in swiss prot under accession No: 11226

An aspect of the invention relates to the use of a composition comprising at least one mannan-binding lectin (MBL) subunit, or at least one mannan-binding lectin (MBL) oligomer comprising the at least one mannan-binding lectin (MBL) subunit, in the manufacture of a medicament for prophylaxis and/or treatment of infection in
a) an individual having an immunocompromised condition caused by a solid tumor or a haematological cancer or/and medical treatment of said tumor or cancer and/or
b) an individual being at risk of acquiring an immunocompromised condition resulting from a solid tumor or a haematological cancer or/and medical treatment of said tumor or cancer.

The MBL composition used to manufacture an MBL medicament may be produced from any MBL source available. The MBL source may be natural MBL, whereby the MBLs are produced in a native host organism, meaning that MBL is produced by a cell normally expressing MBL. One usual method of producing an MBL composition is by extraction of MBL from human body liquids, such as serum or plasma, but MBL may also be harvested from cultures of hepatocytes.

In another aspect the MBL oligomers are produced by a host organism not natively expressing an MBL polypeptide, such as by recombinant technology.

In a first embodiment the MBL source may be serum, from which an MBL composition is obtained by purification from serum, plasma, milk product, colostrum or the like by a suitable purification method, such as affinity chromatography using carbohydrate-derivatised matrices, such as mannose or mannan coupled matrices. Such a method is discussed in WO99/64453, wherein the purification process is followed by a virus-removal step in order to remove infectious agents from the MBL source, since one of the major problems with proteins purified from body liquids is the risk of introducing infectious agents in combination with the desired protein. WO99/64453 is hereby incorporated by reference.

The MBL composition used to manufacture an MBL medicament preferably comprises MBL oligomers having a size distribution substantially identical to the size distribution of MBL in serum, such as a size distribution profile at least 50% identical to the size distribution profile of MBL in serum. By identical is meant that at least 50% of the oligomers has an apparent molecular weight higher than 200 kDa, when analysed by SDS-PAGE and/or Western blot.

In a more preferred embodiment the size distribution profile is at least 75% identical to the size distribution profile of MBL in serum, such as at least 90% identical to the size distribution profile of MBL in serum, and more preferred at least 95% identical to the size distribution profile of MBL in serum.

When purifying from an MBL source initially having another size distribution profile it is preferred that the affinity chromatography used to purify from the MBL source favours purification of oligomers having an apparent molecular weight higher than 200 kDa. This is obtained by using a carbohydrate-derivatized matrix having substantially no affinity to subunits and/or dimers of MBL. Preferably the carbohydrate-derivatized matrix has affinity for substantially only tetrameric, pentameric and/or hexameric recombinant MBLs.

The matrix may be derivatized with any carbohydrate or carbohydrate mixture whereto MBL binds and for which binding of the higher oligomers of MBL are favoured. The carbohydrate-derivatized matrix is preferably a hexose-derivatized matrix, such as a mannose- or an N-acetyl-glucosamin derivatized matrix, such as most preferably a mannose-derivatized matrix.

The selectivity of the carbohydrate-derivatized matrix is obtained by securing that the matrix as such, i.e. the un-derivatized matrix has substantially no affinity to MBL polypeptides, in particular no affinity to MBL trimers or smaller oligomers. This may be ensured when the matrix as such is carbohydrate-free. In particular the matrix should not contain any Sepharose or the like. It is preferred that the matrix consists of a non-carbohydrate containing polymer material, such as Fractogel®TSK beads

The matrix may be in any form suitable for the chromatography, mostly in the form of beads, such as plastic beads.

After application of the MBL source the column is washed, preferably by using non-denaturing buffers, having a composition, pH and ionic strength resulting in elimination of proteins, without eluting the higher oligomers of MBL. Such as buffer may be TBS. Elution of MBL is performed with a selective desorbing agent, capable of efficient elution of higher oligomers of MBL, such as TBS comprising a desorbing agent, such as EDTA (for example 5 mM EDTA) or mannose (for example 50 mM mannose), and MBL oligomers are collected. Such a purification method is described in International patent application No. WO 00/70043 having the title “Recombinant Human Mannan Binding Lectin”.

In a preferred aspect a clinical grade MBL composition is obtained by using an MBL source produced by recombinant technology, wherein the MBL source is the culture media from culturing of MBL producing cells.

Thus, the present invention encompasses MBL produced by a process of producing a recombinant mannan binding lectin (MBL), comprising the steps of:
preparing a gene expression construct comprising a DNA sequence encoding a MBL polypeptide or a functional equivalent thereof,
transforming a host cell culture with the construct,
cultivating the host cell culture, thereby obtaining expression and secretion of the polypeptide into the culture medium, followed by
obtaining a culture medium comprising human recombinant MBLs.

The culture medium comprising the human recombinant MBL polypeptides may then be processed as described above for purification of MBL.

The MBL polypeptide is preferably a mammalian MBL polypeptide, such as more preferably a human MBL polypeptide. The gene expression construct may be produced by conventional methods known to the skilled person, such as described in U.S. Pat. No. 5,270,199.

In another embodiment the gene expression construct is prepared as described in Danish Patent application No: PA 1999 00668 or in co-pending International patent application WO 00/70043.

The expression is preferably carried out in e.g. mammalian cells, the preparation according to the invention results from the use of an expression vector comprising intron sequence(s) from an MBL gene and at least one exon sequence. Regarding the transgenic animals as expression system this term is in this context animals which have been genetically modified to contain and express the human MBL gene or fragments or mimics hereof.

In addition to the purification method it is preferred that the gene expression construct and the host cell also favours production of higher oligomers, which has been found to be possible by using a gene expression construct comprising at least one intron sequence from the human MBL gene or a functional equivalent thereof.

In particular the MBL composition is used for treatment and/or prophylaxis of an infection associated with an immunocompromised condition in an individual, where the condition may be caused by a solid tumor or a haematological cancer or the treatment of such disease. Any microbiological infections may be treated and/or prevented with MBL, i.e. any infection caused by a microbial species.

Consequently, the MBL composition may be used for preventing and/or treating an infection in an immunocompromised individual wherein the microbial species is a fungus, a yeast, a protozoa and/or a bacteria. Particular the fungi species Candida sp. and Aspergillus sp. is considered.

Also, the MBL composition may be used for treating infection, wherein the microbial species is resistant to usual medicaments, such as infections for which the bacterial species is resistant to at least one antibiotic medicament. More important is the prophylaxis and/or treatment of infections for which the bacterial species is multi-resistant.

The immunocompromised individuals may suffer from infections caused by pathogenic bacterial species, such as Streptococcus pneumonia, Salmonella and Staphylococcal species.

It is however well-known that in particular immunocompromised individuals also often suffer from infections caused by bacterial species, that are normally non- pathogenic, i.e. opportunistic pathogens, e.g. E. coli species, and many of these species are resistant to usual antibiotic treatment.

The infection associated with the immunocompromised condition may also be a viral infection, such as a viral infection wherein the virus is a retrovirus. Virus infection caused by influenza virus or virus infections caused by Herpex simple viruses may be associated with the immunocompromised condition. Further virus includes SARS virus (Severe Acute Respiratory Syndrome virus).

Also, the immunocompromised condition may be an infection with the retrovirus Human Immunodeficiency Virus (HIV). However, the viral infections treated and/or prevented according to the invention are normally not caused by a retrovirus, but may for example be caused by a DNA virus.

The medicament may be produced by using the eluant obtained from the affinity chromatography as such. It is however preferred that the eluant is subjected to further purification steps before being used.

In addition to the MBL oligomers, the medicament may comprise a pharmaceutically acceptable carrier substance and/or vehicles. In particular, a stabilising agent may be added to stabilise the MBL proteins. The stabilising agent may be a sugar alcohol, saccharides, proteins and/or amino acids. Examples of stabilising agents may be maltose or albumin.

Other conventional additives may be added to the medicament depending on administration form for example. In one embodiment the medicament is in a form suitable for injections. Conventional carrier substances, such as isotonic saline, may be used.

In another embodiment the medicament is in a form suitable for pulmonal administration, such as in the form of a powder for inhalation or creme or fluid for topical application.

The route of administration may be any suitable route, such as intravenously, intramusculary, subcutaneously or intradermally. Also, pulmonal or topical administration is envisaged by the present invention.

The MBL composition may also be administered simultaneously, sequentially or separately with another treatment, said other treatment resulting in an immunocomprosmising condition in the individual, such as chemotherapy. The medicament may be administered for a period before the onset of administration of chemotherapy or the like and during at least a part of the chemotherapy.

The MBL composition is administered in suitable dosage regimes, in particularly it is administered repeatedly at suitable intervals, such as once or twice a week, starting before onset of chemotherapy and maintained at intervals, for example once a week, at least during a part of the chemotherapy period, preferably during the whole chemotherapy period.

Normally from 1-100 mg is administered per dosage, such as from 2-10 mg, mostly from 5-10 mg per dosage depending on the individual to be treated, for example about 0.1 mg/kg body weight is administered.

The use of an MBL composition may also be in a kit-of-parts further comprising another medicament, such as an anti-fungal, anti-yeast, anti-bacterial and/or anti-viral medicament.

The anti-viral medicament may be a medicament capable of virus attenuation and/or elimination.

The invention also relates to an aspect of using a measurement of the MBL level as a prognostic marker for the risk of the individual of acquiring an infection and thereby an indicative of the need for treatment. In particular an MBL level below 500 ng/ml is a prognostic marker indicative for treatment with MBL, in particular in relation to an individual having a solid tumor or a haematological cancer, whereby the individual is immunocompromised or at risk of being immuncompromised due to treatment of said tumor.

The prognostic marker may be in relation to any infection, but is especially relevant as a prognostic marker for septicaemia or pneumonia in individuals undergoing immunocompromising cell toxic therapy or surgical treatment, such an individual receiving treatment of a solid tumor or a haematological cancer.

Thus, the present invention also relates to a method of using an MBL composition for preventing and/or reducing infections in an individual having a solid tumor or a haematological cancer, the method comprising the steps of:
i) determining serum levels of MBL in an individual,
ii) estimating the probability of the occurrence of a significant clinical infection in the individual, and optionally,
   administering an MBL composition to the individual.

The MBL level is measured in serum or plasma, and may be determined by time resolved immunofluorescent assay (TRIFMA), ELISA, RIA or nephelometry.

Also the MBL levels may be inferred from analysis of genotypes of the MBL genes as discussed above in relation to mutations of MBL leading to a decreased MBL level.

### EXAMPLE

Assay for MBL

The concentration of MBL may be determined by a time resolved immunofluorescent assay (TRIFMA). Microtitre wells (fluoroNunc, Nunc, Kamstrup, Denmark) are coated with antibody by incubation overnight at room temperature with 500 ng anti-human MBL antibody (Mab 131-1, Statens Serum Institut, Copenhagen, Denmark) in 100 μl PBS (0.14 M NaCl, 10 mM phosphate, pH 7.4). After washing with Tween-containing buffer (TBS, 0.14 M NaCl, 10 mM Tris/HCl, 7.5 mM NaN₃, pH 7.4 with 0.05% Tween 20) test samples (plasma 1/20) and calibrator dilutions are added in TBS/Tween with extra NaCl to 0.5 M and 10 mM EDTA.

After overnight incubation at 4° C. and wash, the developing europium-labelled anti-body (12.5 ng Mab 131-1 labelled with the Eu-containing chelate, isothiocyanato-benzoyl-diethylene-triamine-tetra acetic acid, according to the manufacturer, Wallac, Turku, Finland) are added in TBS/Tween with 25 μM EDTA.

Following incubation for 2 h and wash, fluorescence enhancement solution is added (Wallac) and the plates were read on a time resolved fluorometre (Delfia 1232, Wallac). The calibration curve is made using dilutions of one standard plasma, which is kept alliquoted at −80° C. The concentration of MBL in this plasma is determined by comparison with highly purified MBL, which is quantified by quantitative amino acid analysis.

### References

1. Weis W I, Taylor M E and Drickamer K (1998) The C-type lectin superfamily in the immune system. Immunological Reviews 163:19-34
2. Holmskov, U., Malhotra, R., Sim, R. B., and Jensenius, J. C. (1994) Collectins: collagenous C-type lectins of the innate immune defense system. Immunol. Today 15:67-74.
3. Turner, M. W. (1996) Mannose-binding lectin: the pluripotent molecule of the innate immune system. Immunol. Today 17:532-540.
4 Janeway C A, Travers P, Walport M and Capra J D (1999) Immunobiology, the immune system in health and disease, Fourth Edition, Churchill Livingstone)
5. Matsushita, M. and Fujita, T (1992). Activation of the classical complement pathway by mannose-binding protein in association with a novel C1s-like serine protease. J. Exp. Med. 176:1497-1502.
6. Thiel S, Vorup-Jensen T, Stover C M, Schwaeble W, Laursen S B, Poulsen K, Willis A C, Eggleton P, Hansen S, Holmskov U, Reid K B and Jensenius J C (1997) A second serine protease associated with mannan-binding lectin that activates complement. Nature, 386(6624): 506-510
7. Stover C M, Thiel S, Thelen M, Lynch N J, Vorup-Jensen T, Jensenius J C and Schwaeble W J (1999) Two constituents of the initiation complex of the mannan-binding lectin activation pathway of complement are encoded by a single structure gene. J Immunol 162: 3481-3490
8. Thiel S, Holmskov U, Hviid L, Laursen S B and Jensenius J C (1992) The concentration of the C-type lectin, mannan-binding protein, in human plasma increases during an acute phase response. Clin Exp Immunol 90: 31-35
9. Madsen, H. O., Garred, P., Kurtzhals, J. A., Lamm, L. U., Ryder, L. P., Thiel, S., and Svejgaard, A. (1994) A new frequent allele is the missing link in the structural poly-morphism of the human mannan-binding protein. Immunogenetics 40:37-44.
10. Summerfield J A, Ryder S, Sumiya M, Thursz M, Gorchein A, Monteil M A and Turner M W (1995) Mannose binding protein gene mutations associated with unusual and severe infections in adults. Lancet 345: 886-889
11. Garred P, Madsen H O, Hofmann B and Svejgaard A (1995) Increased frequency of homozygosity of abnormal mannan binding protein alleles in patients with suspected immunodeficiency. Lancet 346: 941-943
12. Summerfield J A, Sumiya M, Levin M and Turner M W (1997) Association of mutations in mannose-binding protein gene with childhood infection in consecutive hospital series. BioMed J 314: 1229-1232
13. van Emmerik, L C, Kuijper, E J, Fijen, C A P, Dankert, J, and Thiel, S (1994) Binding of mannan-binding protein to various bacterial pathogens of meningitis. Clin. Exp. Immunol. 97:411-416.
14. Jack D L, Dodds A W, Anwar N, Ison C A, Law A, Frosch M, Turner M W and Klein N J (1998) Activation of complement by Mannose-binding lectin on isogenic mutants of Neisseria meningitidis seroproup B. J Immunol 160: 1346-1353
15. Miller, M. E., Seals, J., Kaye, R., and Levitsky, L. C. (1968) A familial, plasma-associated defect of phagocytosis. A new case of recurrent bacterial infections. The Lancet :60-63.
16. Super, M., Thiel, S., Lu, J., Levinsky, R. J., and Turner, M. W. (1989) Association of low levels of mannan-binding protein with a common defect of opsonisation. Lancet 2:1236-1239.
17. Nielsen, S. L., Andersen, P. L., Koch, C., Jensenius, J. C., and Thiel, S. (1995) The level of the serum opsonin, mannan-binding protein in HIV-1 antibody-positive patients. Clin. Exp. Immunol. 100:219-222.
18. Christiansen, O. B., Kilpatrick, D. C., Souter, V., Varming, K., Thiel, S., Jensenius, J. C. (1999) Mannan-binding lectin deficiency is associated with unexplained recurrent miscarriage. Scand. J. Immunol., 49, 193-196
19. Garred, P, Harboe, M, Oettinger, T, Koch, C, and Svejgaard, A (1994) Dual role of mannanbinding protein in infections: Another case of heterosis ? Eur. J. Immunogen. 21:125-131.
20. Hoal-Van Helden E G, Epstein J, Victor T C, Hon D, Lewis L A, Beyers N, Zurakowski D, Ezekowitz A B, Van Helden P D (1999) Mannose-binding protein B allele confers protection against tuberculous meningitis. Pediatr Res 45:459-64
21. Fischer, P B, Ellerman-Eriksen, S, Thiel, S, Jensenius, J C, and Mogensen, S C (1994) Mannan-binding protein and conglutinin mediate enhancement of herpes simplex virus type-2 infection in mice. Scand J Immunol 39:439-445.
22. Valdimarsson H, Stefansson M, Vikingsdottir T, Arason G J, Koch C, Thiel S and Jensenius J C (1998) Reconstitution of opsonizing activity by infusion of mannan-binding lectin (MBL) to
24. Pizzo, P A (1993), Management of fever in patients with cancer and treatment-induced neutropenia, N Eng J Med, 328, 1323-1332.
25. Aiftoniemi, J., Miettinen, A., Laine, S., Sinisalo, M., Laippala, P., Vilpo, L, Vilpo, J. (1999), Opsonising immunoglobulins and mannan-binding lectin in chronic lymphocytic leukemia, Leuk Lymphoma July; 34(34):3815
26. Lehrnbecher T, Venzon D, de Haas M, Chanock S J, Kuhl J. (1999) Assessment of measuring circulating levels of interleukin6, interleukin8, Creactive protein, soluble Fc gamma receptor type III, and mannosebinding protein in febrile children with cancer and neutropenia. Clin Infect Dis, August; 29(2):4149.
27. Lu, J., Thiel, S., Wiedemann, H., Timpl, R. & K. B. M. Reid (1990) Binding of the pentamer/hexamer forms of mannan-binding protein to zymosan activates the proenzyme C1r₂C1s₂ complex, of the classical pathway of complement without involvement of C1q. J. Immunol. 144:2287-2294.
28. Sastry, K., Herman, G. A., Day, L., Deignan, E., Bruns, G., Morton, C. C. & R. A. B. Ezekowitz (1989) The human mannose-binding protein gene. J. Exp. Med. 170:1175-1189.
29. Lipscombe, R. J., Sumiya, M., Summerfield, J. A. & M. W. Turner (1995) Distinct physicochemical characteristics of human mannose-binding protein expressed by individuals of differing genotype. Immunology 85:660-667.

1-69. (canceled)

70. A method for treatment or reducing the risk of infection in an individual in need thereof comprising administering a composition comprising at least one mannan-binding lectin (MBL) subunit, or at least one mannan-binding lectin (MBL) oligomer comprising the at least one mannan-binding lectin (MBL) subunit to said individual, wherein said individual is;
a) an individual having an immunocompromised condition caused by a solid tumor or/and medical treatment of said tumor and/or
b) an individual being at risk of acquiring an immunocompromised condition resulting from a solid tumor or/and medical treatment of said tumor.

71. The method according to claim 70, wherein the solid tumor is selected from the group consisting of;
female cancers, male cancers, cancers of the respiratory system, cancers of the gastro intestinal system, cancers of the renal system, cancers of the endocrine system and melanomas.

72. The method according to claim 70, wherein the composition comprises at least one mannan-binding lectin (MBL) oligomer comprising the at least one mannan-binding lectin (MBL) subunit.

73. The method according to claim 72, wherein said oligomer is selected from the group of oligomers consisting of tetramers, pentamers and/or hexamers.

74. The method according to claim 70, wherein the individual has a serum level of MBL in excess of 50 ng/ml serum.

75. The method according to claim 74, wherein the serum MBL level is the functional serum MBL level.

76. The method according to claim 70, wherein the medical treatment is surgery.

77. The method according to claim 70, wherein the infection is an infection caused by a microbial species.

78. The method according to claim 77 wherein the microbial species is selected from the group consisting of: a fungus, a yeast, a bacterium and a virus.

79. The method according to claim 78, wherein the microbial species is selected from the group consisting of: Candida sp., Aspergillus sp, a bacterial species resistant to at least one antibiotic medicament, a multi-resistant bacterial species, a pathogenic bacterial species, Herpes Simplex virus, a influenza virus, SARS, a retrovirus and a Human Immunodeficiency Virus.

80. A kit-of-parts comprising a composition comprising at least one mannan-binding lectin (MBL) subunit, or at least one mannan-binding lectin (MBL) oligomer comprising the at least one mannan-binding lectin (MBL) subunit further comprising an antimicrobial medicament capable of attenuation and/or elimination a microbial species.

81. The method according to claim 70, wherein the MBL subunit or the MBL oligomer is produced in a native host organism or by a host organism not natively expressing an MBL polypeptide.

82. The method according to claim 70, wherein the MBL subunit or the MBL oligomer is produced by a method comprising at least one step of recombinant DNA technology in vitro.

83. The method according to claim 82, wherein the production of the MBL subunit or the MEL oligomer is controlled by an expression control sequence not natively associated with MEL polypeptide expression.

84. The method according to claim 81, wherein the MEL subunit or the MEL oligomer is isolated from the host organism by a method comprising at least one step involving affinity chromatography.

85. The method according to claim 84, wherein the affinity chromatography step is capable of isolating MBL tetramers, pentamers and/or hexamers from a composition further comprising additional MBL oligomers and/or MBL subunits.

86. The method according to claim 70, wherein the MBL subunit is a mammalian MBL subunit.

87. The method according to claim 86, wherein the mammalian MBL subunit is a human MBL subunit.

88. The method according to claim 70, wherein the medicament is administered to the individual prior to, during and/or after another medical treatment resulting in an immunocompromising condition in the individual.

89. The method according to claim 88, wherein said medical treatment is surgery.

90. The method according to claim 70, wherein the medicament is a booster of MBL serum levels in an individual having MBL serum levels above a predetermined minimum MBL serum level or wherein the individual has MBL serum levels below a predetermined maximum MBL serum level.

91. The method according to 90, wherein the individual has serum levels of MBL in excess of 75 ng/ml or wherein the individual has serum levels of MBL below 500 ng/ml.
